(19) United States
(12) Patent Application Publication
(10) Pub. No.: US 2025/0025508 A1
(43) Pub. Date: Jan. 23, 2025

(54) COMPOSITIONS AND METHODS FOR TREATING EXPOSURE OF SURGICAL MESH

(71) Applicant: MAYO FOUNDATION FOR MEDICAL EDUCATION AND RESEARCH, Rochester, MN (US)

(72) Inventors: Atta Behfar, Rochester, MN (US); John A. Occhino, Rochester, MN (US); Emanuel C. Trabuco, Rochester, MN (US)

(21) Appl. No.: 18/714,314

(22) PCT Filed: Nov. 30, 2022
(86) PCT No.: PCT/US2022/051424

§ 371 Date: May 29, 2024

Related U.S. Application Data
(60) Provisional application No. 63/284,987, filed on Dec. 1, 2021.

(51) Int. Cl. A61K 35/19; A61K 38/39; A61P 41/00

(52) U.S. Cl. CPC A61K 35/19; A61K 38/39; A61P 41/00

(57) ABSTRACT

A method for treating exposure of surgical mesh in a subject generally includes administering PEP to tissue adjacent to an area of exposed surgical mesh in an amount effective to treat the area of exposed surgical mesh. In another aspect, a method for treating exposure of surgical mesh in a subject generally includes surgically closing an area of exposed surgical mesh and administering PEP to epithelium adjacent to the surgical closure in an amount effective to treat the area of exposed surgical mesh. In one or more embodiments of either aspect, the amount effective to treat the area of exposed surgical mesh can be an amount effective to increase proliferation of epithelium to decrease the area of exposed surgical mesh, increase thickness of epithelium covering the surgical mesh, or increase vascularization of epithelium covering the surgical mesh.

### CROSS-REFERENCE TO RELATED APPLICATIONS

This application claims the benefit of U.S. Provisional Application Ser. No. 63/284,987, filed on Dec. 1, 2021, which is incorporated by reference herein in its entirety.

### GOVERNMENT FUNDING

This invention was made with government support under TR002377 awarded by the National Center for Advancing Translational Studies, National Institutes of Health. The government has certain rights in the invention.

### SUMMARY

This disclosure describes, in one aspect, a method for treating exposure of surgical mesh in a subject. Generally, the method includes administering PEP to tissue adjacent to an area of exposed surgical mesh in an amount effective to treat the area of exposed surgical mesh.

In another aspect, this disclosure describes a method for treating exposure of surgical mesh in a subject. Generally, the method includes surgically closing an area of exposed surgical mesh and administering PEP to epithelium adjacent to the surgical closure in an amount effective to treat the area of exposed surgical mesh.

In one or more embodiments of either aspect, the amount effective to treat the area of exposed surgical mesh can be an amount effective to increase proliferation of epithelium to decrease the area of exposed surgical mesh, increase thickness of epithelium covering the surgical mesh, or increase vascularization of epithelium covering the surgical mesh.

In one or more embodiments of either aspect, the PEP is administered once. In other embodiments, the PEP is administered more than once.

In one or more embodiments of either aspect, PEP is administered in an amount effective to deliver at least 10¹² PEP exosomes.

In one or more embodiments of either aspect, the surgical mesh is at least 1 cm by 1 cm in size.

In one or more embodiments of either aspect, the method results in mesh exposure resolution as measured by increased cellular proliferation, increased epithelial thickness, increased vascularization, or lack of dehiscence compared to an untreated mesh exposure. In one or more embodiments of either aspect, the method results in reduced scar formation as measured by issue microscopy as compared to an untreated mesh exposure.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Experimental methods: creation of the model and intervention. (A) Creation of the mesh exposure model. Day 0: Denude vagina and implant mesh. All animals underwent a primary surgery to create the mesh model. The vagina was denuded according to the planned mesh implant size. The mesh was affixed with sutures. Group 1 had two 3×3 centimeter (cm) meshes implanted on the ventral and rostral vagina. Group 2 had three meshes of different sizes implanted circumferentially: 1×2 cm, 2×2 cm, and 3×3 cm. One animal in this group, which was given EdU, had two 3×3 cm implants rather than different sized meshes. (B) Intervention on Day 7: surgical closure. The four animals in Group 1 underwent a second surgery to mobilize their vaginal epithelium and close vaginal tissues over the underlying mesh in an interrupted fashion (left). Half of these animals receive a concomitant PEP injection (Group 1B). The three animals in Group 2 received an injection of PEP only (right; Group 2). Abbreviations: PEP, purified exosome product. EdU, 2′-Deoxy-5-ethynyluridine

FIG. 2. Gross pathology and transmission electron microscopy (TEM). (A) Gross vagina-mesh complexes, four weeks after initial surgery. Panel 1: wildtype, un-instrumented porcine vagina. Panel 2: Group 1A, surgical closure-only group. Panel 3: Group 1B, PEP injection with surgical closure. Panel 4: Group 2, PEP-injection-only. White dashed boxes represent areas of underlying mesh. All meshes pictured are 3×3 cm. (B) Transmission Electron Microscopy: The wildtype represents healthy pig vagina at the epithelial level (top; 1500× magnification) and deeper into the dermis, with focus at the level of the basement membrane (middle and bottom; 3,000× and 12,000×). Group 1A, who underwent surgical closure-only, had flattened epithelial cells with disruption of cell organization adjacent to an intermittently disrupted basement membrane (center panels). Group 2, who underwent treatment with PEP-only had epithelial cells with normal structure and cell junctions, with some interspersed inflammatory cells (top right, 3000×). The basement membrane and adjacent cell organization are preserved (right, middle and bottom).

FIG. 3. Histologic assessment of vagina-mesh complexes. (A) Representative Masson's Trichrome staining of explants obtained at sacrifice. (B) Representative immunofluorescence images depicting epithelial marker cytokeratin and interstitial collagen III. (C) Representative immunofluorescence images depicting proliferating (Ki67⁺) cells within the epithelium (cytokeratin) and underlying dermal tissues.

FIG. 4. Histologic assessment of vagina-mesh complexes. (A) Quantification of epithelial thickness obtained from Masson's Trichrome staining; ANOVA p=0.03. (B) Quantification of total, epithelial, and non-epithelial proliferating (Ki67⁺) cells per length of epithelium. For (A) and (B), n=2 (wildtype, non-operated vaginal tissue), n=2 (surgical closure only), n=4 (PEP with surgical closure), n=7 for A and n=5 for E (PEP injection only). (C) EdU is present in cells spanning the epithelium and underlying tissue, confirming de novo regeneration during the four-week healing period. Values represented as mean±standard error.

FIG. 5. Cellular analysis of vagina-mesh complexes. (A) Representative immunofluorescence images of CD3⁺ T cells adjacent to E-Cadherin⁺ (E-Cad) epithelium. (B) Representative immunofluorescence images depicting vascularization (CD31⁺ endothelial cells surrounded by smooth muscle actin (SMA⁺) smooth muscle cells), as well as presence of myofibroblasts (extravascular SMA⁺ regions).

FIG. 6. Cellular analysis of vagina-mesh complexes. (A) Quantification of T cell abundance by relative CD3⁺ fraction per high-power view. (B) Quantification of vascularization (CD31⁺ lumens per mm²); ANOVA p=0.03, *p<0.05 relative to wildtype. (C) Quantification of smooth muscle deposition (SMA⁺ area fraction per high-power field) within vagina-mesh explants. (D) Quantification of co-distribution of CD31 and SMA as measured by intensity correlation quotient. Wildtype, non-operated vaginal tissue: n=2; Surgical closure only: n=2; PEP with surgical closure: n=4; PEP injection only: n=5 for A, C, and D; n=7 for B.

FIG. 7. Study Flow Diagram. (A) To create the mesh exposure model, 3 cm by 3 cm areas of epithelium and fibromuscular tissue were denuded and squares of polypropylene mesh were affixed. (B) Animals entered either the acute or subacute arm where they underwent one week (acute) or eight weeks (chronic) of healing. Following this period, they received one injection of PEP once (acute-single PEP, subacute-single PEP) or once weekly for four weeks (acute-weekly PEP). Sacrifice occurred four weeks after the first injection of PEP. Abbreviations: PEP, purified exosome product.

FIG. 8. Epithelial regeneration following PEP treatments for mesh exposure. (A) Representative hematoxylin and eosin (H&E) images of explanted mesh-vagina complexes. #denotes holes created by 80 μm-thick mesh fibers. The cross-sectional image in the acute-single group depicts two joining fibers, hence the larger diameter hole. (B) Mean thickness of regenerated vaginal epithelium across the various PEP-treated groups, measured over 4±1.4 cm of epithelium per explanted mesh. ANOVA p=0.2. (C) Representative immunofluorescence images of vaginal epithelium (Cytokeratin, green) labeled with EdU (red) as a marker of cell regeneration. (D) Quantification of total, epithelial, and non-epithelial proliferating cell density (EdU⁺ cells per area), measured over 1.6±0.3 cm of epithelium per mesh. ANOVA for total (p=0.049), epithelial (p=0.046), non-epithelial (p=0.043). For B/D, *p<0.05, n=2 (acute-single PEP), n=4 (acute-weekly PEP), and n=6 (subacute-single PEP); data reported as mean±SEM.

FIG. 9. Vascularization of regenerated tissues following varying PEP treatments for mesh exposure. (A) Representative immunofluorescence images of subepithelial blood vessels (CD31, red), with EdU (white) marking regenerated cells. (B) Quantification of capillary density reported as CD31⁺ lumens per area of sub-epithelium (8.5±1.2 mm² per mesh). ANOVA for vessels (p=0.0069), EdU⁺ vessels (p=0.022); *p<0.05, **p<0.01, n=2 (acute-single PEP), n=4 (acute-weekly PEP), and n=6 (subacute-single PEP); data reported as mean±SEM.

### DETAILED DESCRIPTION OF ILLUSTRATIVE EMBODIMENTS

Surgical implantation of polypropylene mesh is a widely accepted and durable treatment for women experiencing, for example, stress urinary incontinence or pelvic organ prolapse. These procedures are often performed if behavioral/non-surgical measures are unsuccessful eliminating symptoms. While implantation of mesh is an effective treatment, some women experience mesh exposure in the vagina over time. Mesh exposure may occur along the suture line or any area overlying the implant; it is suspected to occur due to a combination of intra-operative devascularization during dissection and poor postoperative wound healing. Many factors can contribute to a woman's risk of mesh exposure, including the volume of mesh implanted, nutritional status, menopausal status, and medical comorbidities such as diabetes, immunosuppression, and cigarette smoking. Persistent exposure of mesh can cause dyspareunia, psychological distress, abnormal discharge, and predispose women to vaginal infection. While vaginal estrogen is often used as a non-surgical therapy for exposure, high-level data on efficacy does not exist and many patients go on to require reoperation for epithelial re-closure or mesh resection. Reoperation involves significant cost and resection may lead to return of presenting symptoms.

While described herein in the context of treating mesh exposure, the compositions and methods described herein may be applicable in other contexts such as, for example, during initial implantation or removal of surgical mesh. The compositions described herein may be administered, and/or the methods described herein may be practiced, before a mesh implantation procedure, during a mesh implantation procedure, after a mesh implantation procedure but before any exposure or other complication, as a treatment if mesh exposure occurs, during a mesh removal procedure, immediately after a mesh removal procedure, or at some time after a mesh removal procedure. In addition, while described herein in the context of an exemplary model of vaginal mesh exposure, it should be understood that the compositions and methods described herein may be applicable to other procedures involving surgical mesh such as, for example, hernia repair, treatment of stress urinary incontinence, uterine prolapse, or reinforcement of other damaged tissue.

Research in tissue regeneration has increasingly turned to investigating stem cell therapies. Therapeutic success has been limited, however, by cost and heterogeneity of culture techniques and delivery methods. In contrast, exosome delivery platforms can deliver therapeutic nucleic acids (e.g., miRNAs), signaling proteins, and other regenerative substrates to activate endocrine, paracrine, and/or anti-inflammatory pathways. Exosomes can drive local stem cell activation and proliferation, obviating the need for cell-based modalities.

This disclosure describes therapeutic compositions that include a purified exosome product (PEP). These compositions may be employed in methods for treating vaginal mesh exposure. PEP is a purified exosome product prepared using a cryodesiccation step that produces a product having a structure and composition that is distinct from exosomes prepared using conventional methods. PEP is room-temperature-stable and can be reconstituted in a pharmaceutical carrier (e.g., in pH neutralized Collagen-1), as described in more detail below, to create an injectable composition for nonsurgical management of poor vaginal tissue healing.

Production of purified exosome product (PEP) involves separating plasma from blood, isolating a solution of exosomes from separated plasma with filtration and centrifugation. PEP is fully characterized and methods for preparing PEP are described in International Patent Application No. PCT/US2018/065627 (published as International Publication No. WO 2019/118817), U.S. Patent Publication No. 2021/0169812 A1, and U.S. Pat. No. 10,596,123, each of which is incorporated by reference herein in its entirety. Briefly, PEP is a purified exosome product prepared using a cryodesiccation step that produces a product having a structure that is distinct from exosomes prepared using conventional methods. For example, PEP typically has a spherical or spheroidal structure and an intact lipid bilayer rather than a crystalline structure that results from the reaggregation of lipids of the exosome lipid bilayer after exosomes are disrupted during convention exosome preparation methods. The spherical or spheroid exosome structures generally have a diameter of no more than 300 nm. Typically, a PEP preparation contains spherical or spheroid exosome structures that have a relatively narrow size distribution. In some preparations, PEP includes spherical or spheroidal exosome structures with a mean diameter of about 110 nm±90 nm, with most of the exosome structures having a mean diameter of 110 nm±50 nm such as, for example, 110 nm±30 nm.

An unmodified PEP preparation—i.e., a PEP preparation whose character is unchanged by sorting or segregating populations of exosomes in the preparation—naturally includes a mixture of CD63⁺ and CD63⁻ exosomes. Because CD63⁻ exosomes can inhibit unrestrained cell growth, an unmodified PEP preparation that naturally includes CD63⁺ and CD63⁻ exosomes can both stimulate cell growth for wound repair and/or tissue regeneration and limit unrestrained cell growth.

Further, by sorting CD63⁺ exosomes, one can control the ratio of CD63⁻ exosomes to CD63⁻ exosomes in a PEP product by removing CD63⁺ exosomes from the naturally-isolated PEP preparation, then adding back a desired amount of CD63⁺ exosomes. In one or more embodiments, a PEP preparation can have only CD63⁻ exosomes.

In one or more embodiments, a PEP preparation can have both CD63⁺ exosomes and CD63⁻ exosomes. The ratio of CD63⁺ exosomes to CD63⁻ exosomes can vary depending, at least in part, on the quantity of cell growth desired in a particular application. For example, a CD63⁺/CD63⁻ exosome ratio provides desired cell growth induced by the CD63⁺ exosomes and inhibition of cell growth provided by the CD63⁻ exosomes achieved via cell-contact inhibition. In certain scenarios, such as in tissues where non-adherent cells exist (e.g., blood derived components), this ratio may be adjusted to provide an appropriate balance of cell growth or cell inhibition for the tissue being treated. Since cell-to-cell contact is not a cue in, for example, tissue with non-adherent cells, one may reduce the CD63⁺ exosome ratio to avoid uncontrolled cell growth. Conversely, if there is a desire to expand out a clonal population of cells, such as in allogeneic cell-based therapy or immunotherapy, one can increase the ratio of CD63⁺ exosomes to ensure that a large population of cells can be derived from a very small source.

Thus, in one or more embodiments, the ratio of CD63⁺ exosomes to CD63⁻ exosomes in a PEP preparation may be at least 1:1, at least 2:1, at least 3:1, at least 4:1, at least 5:1, at least 6:1, at least 7:1, at least 8:1, at least 9:1, at least 10:1, at least 11:1, at least 12:1, at least 13:1, at least 14:1, at least 15:1, or at least 16:1. In one or more embodiments, the ratio of CD63⁺ exosomes to CD63⁻ exosomes in a PEP preparation may be at most 15:1, at most 16:1, at most 17:1, at most 18:1, at most 19:1, at most 20:1, at most 25:1, or at most 30:1. For example, the ratio of CD63⁺ exosomes to CD63″ exosomes may be between 1:1 to 30:1, 2:1 to 20:1, 4:1 to 15:1, or 8:1 to 10:1. In one or more certain embodiments, the PEP product is formulated to contain a 9:1 ratio of CD63⁺ exosomes to CD63⁻ exosomes. In one or more certain embodiments, native PEP, e.g., PEP with an unmodified ratio of CD63⁺ exosomes to CD63⁻ exosomes may be used.

This disclosure describes the use of PEP as an adjunct to surgical intervention or as a stand-alone therapy to treat exposure of surgical mesh (e.g., vaginal mesh). The addition of PEP to standard surgical re-closure results in improved resolution—e.g., healing and reclosure of tissue—of mesh exposure in a porcine model. The described porcine model is a valid indicator of clinical utility for use, generally, in various contexts of surgical mesh exposure and in subjects of other species, including humans. Furthermore, PEP can independently achieve mesh exposure resolution without further surgical intervention in clinically meaningful sized (e.g., greater than 1×1 cm) exposures. In keeping with the tenets of regenerative medicine (regeneration of functional tissue), PEP-treated tissue demonstrates neovascularization, epithelial thickness, viscoelastic and inflammatory profiles superior to non-PEP-treated exposures.

While illustrated below in the context of an exemplary model in which the subject is porcine, the compositions and methods described herein may involve any suitable subject. As used herein, a “subject” can be a human or any non-human mammal. Exemplary non-human mammalian subjects include, but are not limited to, a livestock animal or a companion animal. Exemplary non-human animal subjects include, but are not limited to, animals that are hominid (including, for example chimpanzees, gorillas, or orangutans), bovine (including, for instance, cattle), caprine (including, for instance, goats), ovine (including, for instance, sheep), porcine (including, for instance, swine), equine (including, for instance, horses), members of the family Cervidae (including, for instance, deer, elk, moose, caribou, reindeer, etc.), members of the family Bison (including, for instance, bison), feline (including, for example, domesticated cats, tigers, lions, etc.), canine (including, for example, domesticated dogs, wolves, etc.), avian (including, for example, turkeys, chickens, ducks, geese, etc.), a rodent (including, for example, mice, rats, etc.), a member of the family Leporidae (including, for example, rabbits or hares), members of the family Mustelidae (including, for example ferrets), or member of the order Chiroptera (including, for example, bats).

Also, while illustrated below in the context of an exemplary embodiment involving damaged vaginal epithelium as a result of mesh exposure, the compositions and methods described herein may involve treatment of any tissue—and, therefore, any epithelial tissue—damaged as a result of mesh exposure.

### Surgical Mesh Exposure Model

All animals completed the study without adverse events. The described mesh exposure model proved to be adequate to study clinically meaningful sized implants. Swine weighing 70-80 kg allowed for adequate vaginal caliber and depth to implant multiple meshes and for use of standard surgical instruments. All meshes were retained by affixing with 2-0 polypropylene. One animal in the PEP-only group did experience “ballooning” of the central portion of the mesh implants, however still demonstrated re-epithelialization under the mesh (histology confirmed).

### Gross Analysis of Vagina/Mesh Complexes

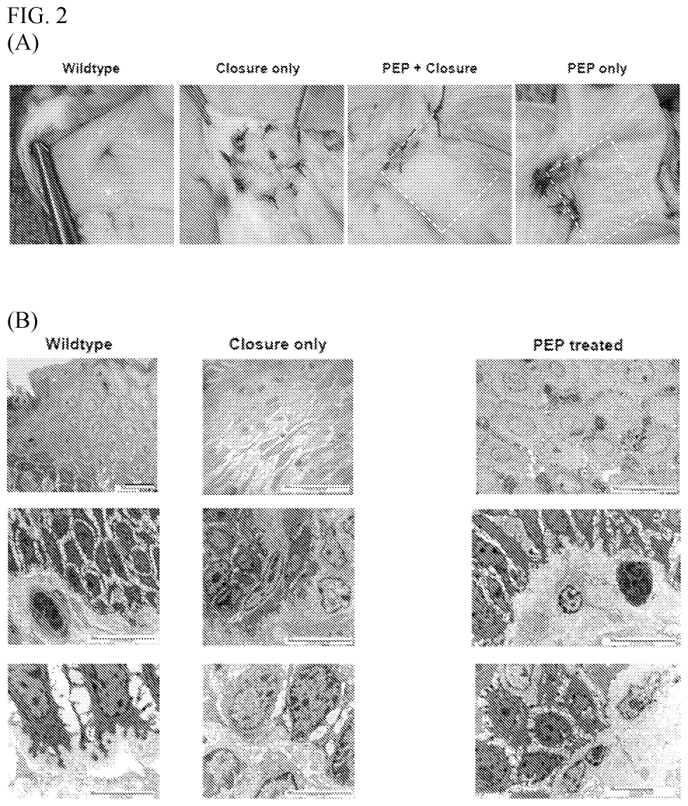

FIG. 2A shows gross examination of the explanted vagina-mesh complexes. Tissues treated with PEP and concomitant surgical closure (Group 1B) demonstrated the most prominent resolution of mesh exposure, followed by the PEP-only (Group 2), then the surgical closure-only group (Group 1A).

Among groups treated with surgical closure, adding PEP decreased the incidence of wound dehiscence (e.g., separation of tissue around the wound) compared to closure-only. All four of the closure-only meshes (Group 1A) had dehiscence and re-exposure of the mesh. The four PEP+closure mesh exposures (Group 1B) demonstrated 50-95% mesh exposure resolution.

PEP-only treated animals (Group 2) demonstrated robust vaginal regeneration despite the lack of surgical closure. One animal had ballooning of the mesh complexes, as previously described. Thus, gross mesh exposure resolution cannot be commented upon. On histology, tissue samples beneath the ballooned meshes showed presence of regenerated tissue central to the previously denuded area. Remaining animals in the PEP-only group showed regeneration of vaginal tissue on the 1 centimeter (cm), 2 cm, and 3 cm implants, with the largest implant demonstrating almost complete resolution of the exposure (FIG. 2A).

### Histological Analysis of Regenerated Tissues

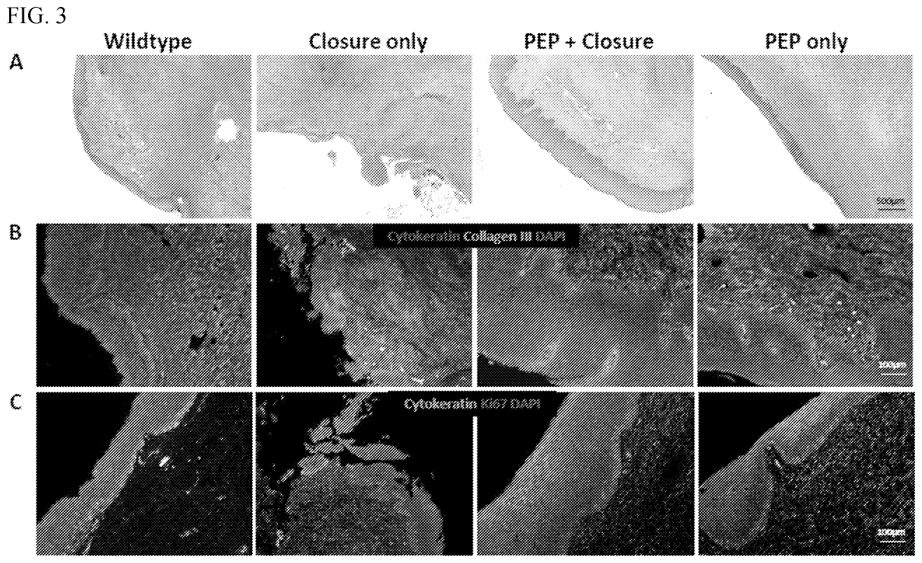

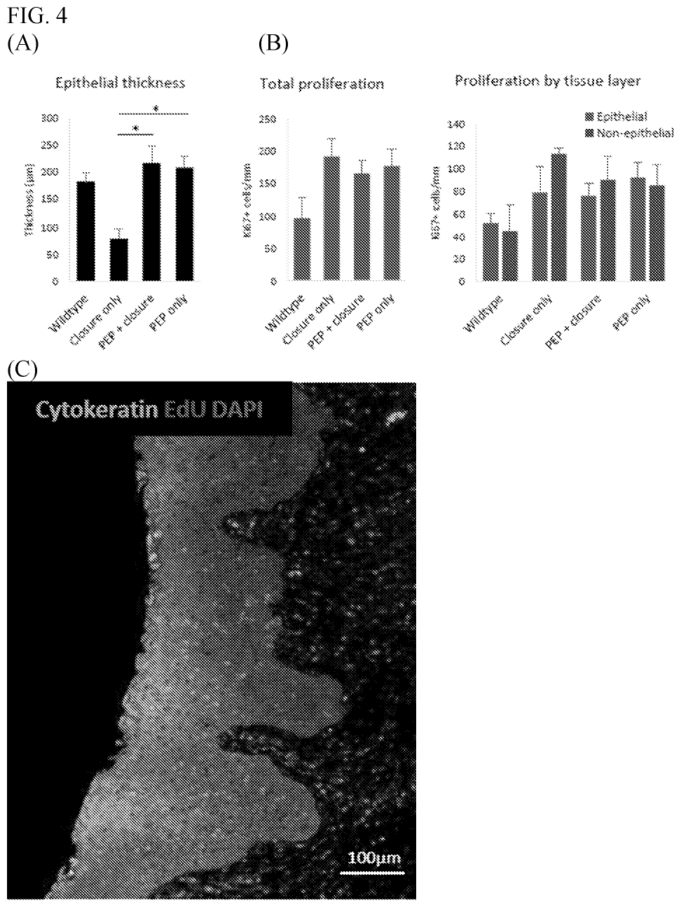

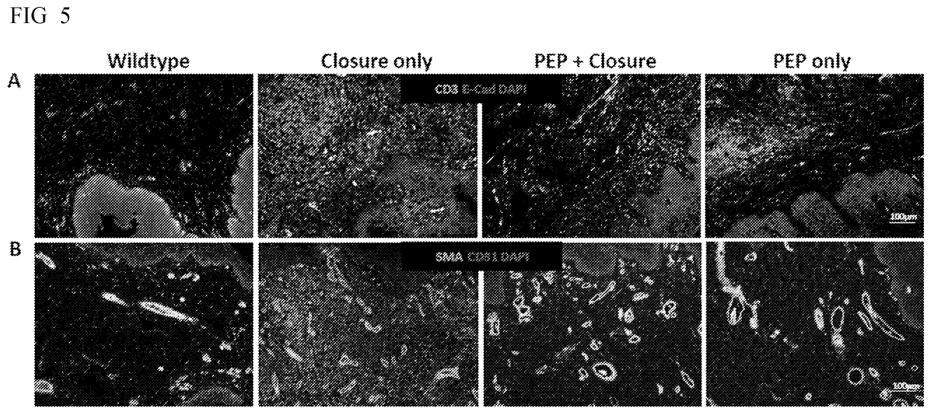

PEP-treated vagina-mesh complexes with or without surgical closure showed re-establishment of a basement membrane (FIG. 2B) and intact epithelium similar to uninjured, wildtype vaginal tissue (FIG. 2A). In contrast, closure-only tissues showed frequent disruptions and cellular disarray in the epithelium (FIG. 2A; FIG. 3A,B). TEM analysis confirmed the presence of a basement membrane and round cells with maintained cell junctions in the PEP+closure group, in contrast to a poorly regenerated basement membrane and scar formation in the closure-only group (FIG. 2B). Histologic specimens were reviewed by an independent, blinded pathologist, corroborating the findings presented herein. Hematoxylin and eosin (H&E)-stained sections revealed increased epithelial thickness in PEP+closure (217 μm) and PEP-only (208 μm) samples as compared to controls (80 μm, p=0.03; FIG. 3A, FIG. 4A). Immunohistochemical analysis of PEP-treated tissues demonstrated appropriate expression of epithelial markers Pan-cytokeratin (FIG. 3B,C), submucosal collagen III (FIG. 3B), and E-Cadherin (FIG. 5A), similar to wildtype tissues.

In one or more embodiments, the methods and compositions described herein may decrease formation of scar tissue at or around surgical mesh. Scar tissue formation may be measured by, for example, microscopic visualization of flattened, disorganized epithelial cells. Non-uniform scar tissue formation can be observed visually at the site of mesh implantation with partial mesh morphology still present (FIG. 2A “Closure only”) as compared to the uniform tissue structure and complete covering in the PEP-treated groups (FIG. 2A “PEP+Closure” and “PEP Only”). Histologically, the abnormal morphology of the epithelial layer can be appreciated in the “Closure only” group (FIG. 2B) compared to the wildtype untreated control group. PEP treatment restored the uniform tissue architecture and cellular organization (FIG. 2B) observed in the wildtype.

### Assessment of Proliferation and Inflammation in Regenerated Tissues

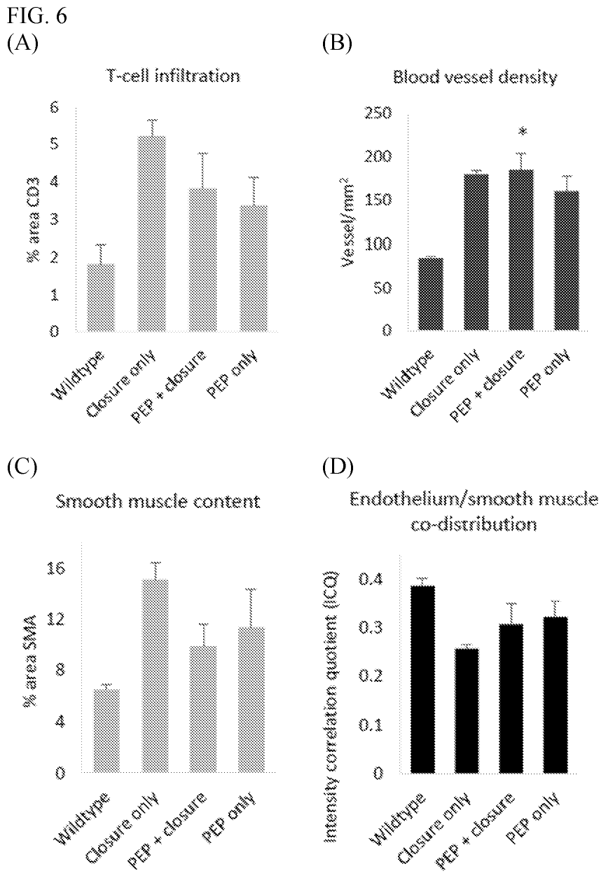

The basal cell layers of squamous epithelium in the vagina are responsible for proliferation. Proliferation was therefore measured by measuring the number of Ki67⁺ cells per length of epithelium. Relative to uninjured wildtype vaginal tissue, all experimental animals showed a trend towards higher rates of total and epithelial proliferation (FIG. 3C; FIG. 4B), with dense Ki67⁺ cells lining the basal layer of the epithelium. To ensure that restored epithelium was derived from new cells, 2′-deoxy-5-ethynyluridine (EdU) patterns were assessed. In the PEP-only group, EdU was present throughout the epithelium and underlying tissues, confirming de novo regeneration during the study period (FIG. 4C). Closure-only vagina/mesh complexes tended to have higher proliferation in the non-epithelial submucosal compartment (FIG. 4B), suggestive of infiltration of rapidly proliferating fibroblasts and/or inflammatory cells. Analysis of CD3 expression demonstrated the highest trend towards T cell infiltration in the closure-only subgroup (FIG. 5A; FIG. 6A), indicating chronic inflammation.

### Vascularization and Fibrosis in Vagina/mesh Complexes

Analysis of endothelial cell presence via CD31⁺ expression demonstrated higher capillary density in the PEP+closure group compared to wildtype tissues (185 vessels/mm² vs. 85 vessels/mm², p=0.03; FIG. 4B; FIG. 6B). Interestingly, closure-only tissues showed increased expression of smooth muscle actin (SMA), in part due to excess interstitial smooth muscle deposition (FIG. 6C). PEP-treated tissues demonstrated increased SMA expression relative to uninjured tissues, but decreased relative to closure-only tissues. Analysis of co-distribution of SMA and CD31 demonstrated the highest intensity correlation quotient (ICQ) in the uninjured wildtype tissues, lowest in the closure-only group, and intermediate in PEP-treated tissues (FIG. 6D) confirming interstitial smooth muscle deposition in closure-only cohorts.

The work described above establishes the utility of PEP injection for resolving acute (e.g., week-old) vaginal mesh exposures in a porcine model of mesh exposure. In the work described below, the effect of re-dosing (single-dose regimen versus multi-dose regimen) on wound healing in a mesh exposure model. Specifically, epithelial thickness, regenerating cellular populations, and neovascularization were evaluated. The multi-dose regimen exhibits the highest regenerative profile. Mesh exposure resolution was compared with surgical intervention, surgical intervention with concomitant PEP injection, and PEP injection-alone. In short, PEP-alone variably treated mesh exposures up to 3 cm by 3 cm in size. PEP-treated tissues had thicker regenerated epitheliums and a lower fibrosis and inflammatory profile as compared to surgical management alone.

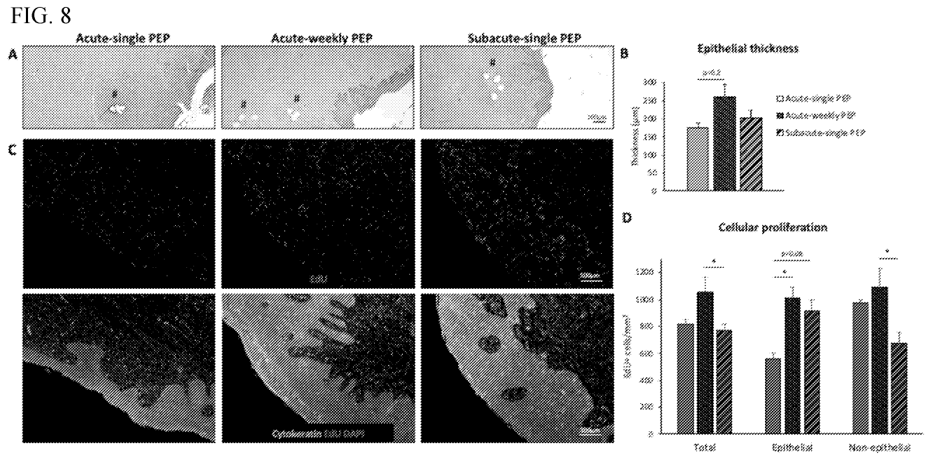

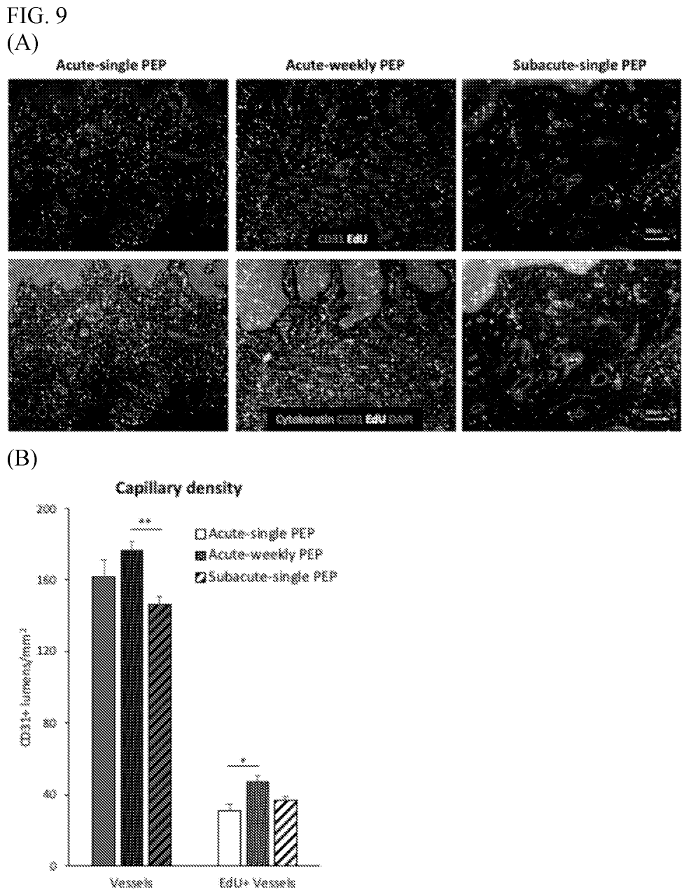

All on-protocol animals completed the study without adverse events. Systematic, multi-organ on and off-target autopsy evaluation was performed, with no concerning findings. Safety evaluation also included multi-organ histologic evaluation, periodic vital signs, and bloodwork. Results were analyzed in aggregate, across all three groups using one-way ANOVA followed by pair-wise comparisons with a post-hoc Tukey's test. Results are presented evaluating (1) single versus multi-dosing protocol, and (2) acute versus subacute mesh exposure (e.g., FIG. 4, FIG. 6, FIG. 8, and FIG. 9).

In one or more embodiments, the method and compositions described herein may improve surgical mesh resolution as measured by any suitable histological measurement. For example, the methods and compositions described herein may increase vascularization, decrease fibrosis, decrease inflammatory profile, increase epithelial thickness, increase cell proliferation, and/or increase expression of markers of tissue repair, such as e-cadherin, cytokeratin, and collagen.

### Single Versus Multi-Dose PEP Regimen in Acute Mesh Exposures

Evaluation of hematoxylin and eosin (H&E)-stained sections revealed increased epithelial thickness (single PEP 175.4 μm, weekly PEP 259 μm, ANOVA p=0.2, not statistically significant) and qualitatively fewer epithelial disruptions in the weekly PEP group, as compared to the single PEP group (FIG. 8A,B). Mesh fibers were noted to be well-integrated in the subepithelial fibromuscular tissue in both groups. The weekly PEP group exhibited fewer peri-fiber nucleated cells.

Since animals were administered EdU twice weekly after their first PEP injection, post-intervention regeneration of the tissues was tracked. Regeneration was reported for total (epithelial and non-epithelial), epithelial, and non-epithelial cellular populations (reported as a density). Total cellular proliferation was significantly different amongst all groups (ANOVA p=0.049, FIG. 8D). Comparing the acute-weekly PEP group to the group receiving a single PEP injection, there was a trend toward higher total proliferation, but this was not significant in post-hoc analysis (p=0.216). Epithelial proliferation was nearly two-fold higher in the acute-weekly PEP group versus the acute-single PEP group (1011 vs. 556 EdU⁺ cells/mm², p=0.038). This indicated that the multi-dosing regimen contributed to a higher proportion of epithelial proliferation, which is further supported by increasing epithelial thickness even though the difference in epithelial thickness did not reach statistical significance.

Analysis of endothelial cell presence using CD31⁺ expression showed a significant difference in capillary density amongst groups (ANOVA p=0.007, FIG. 9B). Post-hoc analysis of capillary density between single versus multiple dosing was not significant (p=0.316). Evaluating regenerating capillary density, as measured by EdU⁺/CD31⁺ vessel lumens/mm², there was a significant difference in regenerating capillary density amongst groups (ANOVA p=0.022), with the weekly PEP group having a higher regenerating capillary density (p=0.030) as compared to the single PEP dose group.

Treatment of Acute Versus Subacute Mesh Exposures with Single PEP Injections

The subacute exposure group exhibited fewer disruptions in the epithelium on hematoxylin and eosin (H&E)-stained sections (FIG. 8A). Notably, mesh fibers were well-integrated in the sub-epithelial space with fewer surrounding nucleated cells in the subacute versus acute group, possibly indicating a decrease in acute-phase inflammatory cells with time. Comparing across acute and subacute mesh exposure groups, epithelial thickness was similar (acute 175 μm vs. subacute 203 μm, p=0.828; FIG. 8B).

Total cellular proliferation between the acute and subacute groups was not statistically different (p=0.943). However, subacute groups exhibited higher epithelial proliferation relative to acute groups (subacute 914 vs. acute 556 EdU⁺ cells/mm², p=0.081, not statistically significant; FIG. 8D), with a higher ratio of epithelial to non-epithelial proliferation (subacute 1.5 vs. acute 0.6). This trend is visually depicted in FIG. 8C, where EdU is concentrated in the cytokeratin positive epithelium.

Capillary density was not different between the acute and subacute mesh exposure groups (p=0.253; FIG. 9B). The regenerating capillary density of the single-dose acute group was similar to the subacute group (p=0.526). Notably, the vessel lumens of the subacute group appeared larger (FIG. 9A), indicating natural vessel coalescence with healing, which partially explains the trend toward decreasing capillary density. Furthermore, comparing the EdU⁺ vessel fraction, rather than density, the subacute group had 25% EdU⁺ vessels compared to 19% in the acute group. This further corroborates the fact that while vessel lumen density is lower, both the absolute number and size of regenerated vessels is higher in the subacute group.

In this preclinical large animal study using an injectable exosome regenerative platform (PEP) in a vaginal mesh exposure model, a multi-dose regimen was found to be superior to a single-dose regimen, and PEP exosomes were effective in both acutely exposed meshes and subacutely exposed meshes. These findings add to data that established the efficacy of PEP as a non-surgical treatment for vaginal mesh exposure. Specifically, this study established patterns of regenerated vaginal tissue and vessels by use of EdU. Exosomes typically deliver a host of growth factors, antioxidants, microRNA, and endocrine/paracrine signals to incite localized regeneration. Data herein showing dose dependent EdU⁺ neovascularization and epithelial proliferation provides further evidence of this mechanism.

Factors including menopausal status, nutritional status, medical comorbidities such as diabetes mellitus and immunosuppression, and cigarette smoking contribute to vaginal mesh exposure. A common thread among these factors is their effect on wound healing. The regenerative properties of PEP exosomes were evaluated for addressing vaginal mesh exposures.

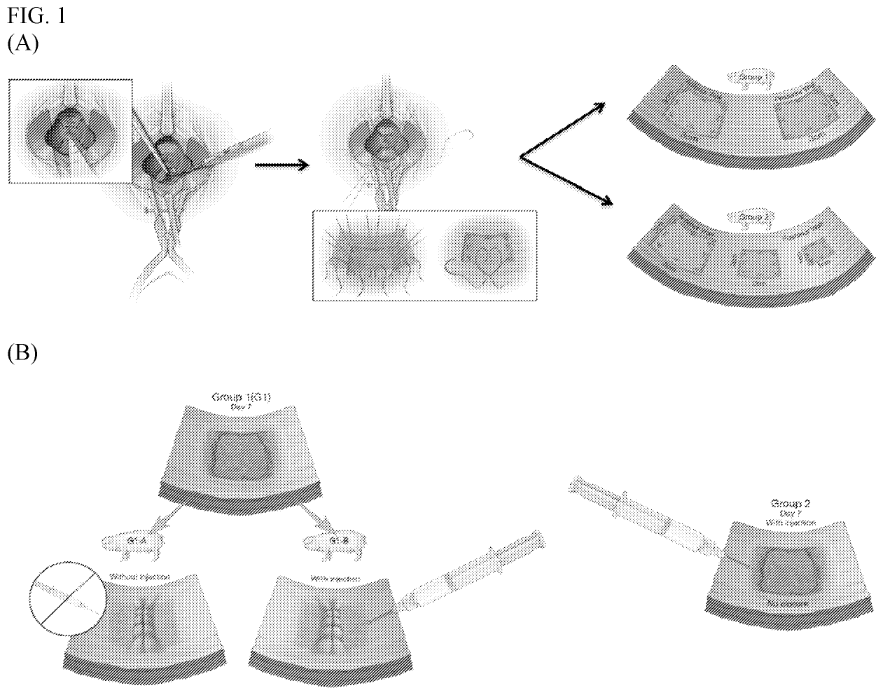

PEP improves to vaginal tissue regeneration after mesh exposure to a degree that suggests that surgery may not be necessary, or could be used as a second-line treatment for mesh exposure (FIGS. 1-6). Further, the effects of re-dosing and timing of PEP administration were evaluated. Regenerated epithelium (thickness and EdU⁺ fractions) and regenerated capillary density were selected as indicators of tissue regeneration.

Epithelial regeneration was used as a measurable marker of vaginal tissue regeneration given the epithelium and fibromuscular tissue were denuded in creation of the model. There was a notable increase in epithelial thickness with repeated dosing. Even a single injection on a delayed schedule produced meaningful regrowth of epithelium. Epithelial regeneration (EdU⁺ epithelial cells/mm²) was statistically higher using the multi-dose regimen, suggestive of a positive dose-response effect. In fact, epithelial regeneration was higher in the single-dose subacute group as compared to the acute group, along with relative decreases in the proliferating fraction within sub-epithelial layers, which may be attributable to capillary coalescence patterns, discussed below.

Regenerated capillary density was higher using the multi-dose protocol yet similar between the single-dose acute and subacute mesh exposures. The maturation phase of neovascularization extends beyond 2-4 weeks post injury. As such, an increase in the number of well-formed lumens at four weeks post-PEP injection is suggestive of improved physiological neovascularization. Looking at the pattern and distribution of vessels on immunohistochemistry (FIG. 9A), the acute groups have more numerous small capillaries, contrasting with the fewer but larger vessels within the subacute group. This pattern of small capillaries coalescing to form mature vessels is well-documented in the wound healing literature. The observed vessel maturation drives on-target proliferation, as seen with the proportion of epithelial/non-epithelial proliferation in this study.

This disclosure presents data from a porcine mesh inlay model that supports the use of a novel exosome therapy, PEP, for surgical mesh (e.g., vaginal mesh) exposures. PEP could therefore serve as a non-operative, cost-saving solution to mesh exposure. PEP is shelf-stable and easy to inject, making it accessible to clinicians in urban and rural practices, as well as for global health outreach. This therapy circumvents issues with heterogeneity of cell culture and cell delivery associated with stem cells. The data presented herein demonstrates a benefit with re-dosing.

Thus, this disclosure describes a model animal study using an injectable acellular regenerative platform for mesh exposure. PEP injection with surgical intervention resulted in robust vaginal tissue regeneration and resolution of mesh exposure compared to surgical intervention alone, all of which experienced dehiscence/re-exposure of mesh. PEP-only treated exposures (no surgical intervention) showed variable mesh exposure resolution, with resolution being achieved up to a size of 3×3 cm. Regenerated tissue established appropriate microanatomy as demonstrated on TEM and light microscopy. Furthermore, PEP-treated tissues showed lower fibroblast proliferation and inflammation. These findings reveal the regenerative, anti-inflammatory, and vasculogenic potential of PEP. Overall, PEP-treated tissues demonstrated thicker regenerated epithelium, with a diminished fibrosis and inflammatory profile compared to the closure-only group. Further, multi-dose regimens are shown to further increase the effects of PEP administration on exposed mesh resolution.

Importantly, the data reported herein complements existing data on chronic unhealed wounds and, more specifically, host response to mesh implantation and drivers of mesh complications. Mesh/vagina complexes in women who have experienced surgery for mesh complications can include tissues with exposures having an exaggerated M2 macrophage and matrix metalloproteinase-9 (MMP) response and relative increase in T cell populations, indicating fibrosis and degradation. In a nonhuman primate model, heavier, lower porosity, and stiffer meshes had deleterious effects on the extracellular matrix, leading to increased MMPs and collagen and elastin catabolismoor host regenerative efforts (e.g., chronic inflammation, ECM remodeling) contribute to mesh exposure and subsequent failure of surgical management of mesh exposures. Thus, using PEP to attenuate inflammation and promote controlled regeneration can decrease the risk of mesh exposure and promote healing in the setting of an existing exposure.

The data presented herein demonstrate that exosome therapies, such as PEP, hold promise in vaginal tissue regeneration. Vaginal mesh exposures treated with exosomes demonstrated robust vaginal tissue regeneration, resulting in partial to full exposure resolution. Application of this platform could result in a paradigm shift in how urogynecologists approach mesh complications.

This disclosure therefore describes compositions and methods for improving repair of mesh exposure. Generally, the compositions include PEP and a pharmaceutically acceptable carrier. In a surgical setting, the PEP may be combined with a carrier that is suitable for application to surgically repaired tissue such as, for example, a surgical glue or a tissue adhesive.

Thus, the method includes administering an effective amount of the composition to a tissue in need of repair following exposure of a surgically implanted mesh. As used herein, a tissue in need of repair can mean tissue adjacent to the surgical mesh such as, for example, degenerated vaginal epithelium and/or vaginal epithelial tissue that is in direct contact with vaginal mesh. Administering the composition to a tissue in need of repair also can include administering the composition to intact or healthy tissue in close enough proximity to the damaged tissue that administration at the intact or healthy site promotes healing of the nearby damaged tissue without further disrupting the damaged tissue with, for example, a needle used to deliver the composition. For example, the PEP composition may be administered by injection to healthy tissue that is up to 25 cm (e.g., 1 cm, 2 cm, 3 cm, 4 cm, 5 cm, 10 cm, 20 cm, etc.) from the damaged tissue.

In this aspect, an “effective amount” is an amount effective to increase epithelial thickness, proliferation of epithelial cells, and/or vascularization (e.g., capillary density) compared to untreated epithelial tissue or epithelial tissue treated with carrier (no PEP) alone. PEP may be formulated with a pharmaceutically acceptable carrier to form a pharmaceutical composition. As used herein, “carrier” includes any solvent, dispersion medium, vehicle, coating, diluent, antibacterial, and/or antifungal agent, isotonic agent, absorption delaying agent, buffer, hydrogel, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the PEP without causing any undesirable biological effects or interacting in a deleterious manner with any of the other components of the pharmaceutical composition in which it is contained. As noted above, in a surgical setting, exemplary suitable carriers include surgical glue, tissue adhesive, or a supportive matrix (e.g., a collagen scaffold).

In one or more embodiments, the PEP composition may include additional components, either in admixture with the PEP vesicles or loaded into at least a portion of the PEP vesicles. In one or more embodiments, the additional components can promote healing. In one or more embodiments, the additional component may include, for example, one or more biocompatible scaffold components such as, for example, collagen, fibrin, fibronectin, laminin, proteoglycan, hyaluronic acid, alginate, or other biocompatible scaffold components. In one or more embodiments, the additional component may include one or more pharmaceutically active ingredients. In one or more embodiments, the pharmaceutically active ingredient may be selected to, for example, decrease healing time, provide pain relief, or reduce the likelihood or severity of a complication (e.g., infection). A pharmaceutically active ingredient may include, for example, a steroid (e.g., estrogen), an analgesic, or an antibiotic. One or more of the additional components may be loaded into at least a portion of the PEP vesicles. The presence of certain components in a PEP preparation may be the result of, for example, expression of the component (e.g., a protein) by the donor cell from which the PEP was derived. Additionally or alternatively, one or more components may be loaded into at least a portion of the PEP vesicles either directly (loading of the component itself) or indirectly (e.g., recombinantly expressed proteins).

In one or more embodiments wherein the composition includes collagen, the collagen may be provided as procollagen, fibrillar collagen, such as type I collagen, type III collagen, or a combination thereof. In embodiments wherein the composition includes collagen, the collagen may be provided as a collagen scaffold. In one or more other embodiments, the extracellular matrix components may be supplied in any suitable form, such as purified recombinant protein. In one or more embodiments, the composition may include PEP and one or more supportive matrix components (e.g., collagen) in a ratio of 1:20 to 1:5 (5% v/v to 20% v/v). In one or more alternative embodiments, the ratio of PEP to supportive matrix components may be 1:100, 1:500, 1:1000, 1:10, 1:5, 1:2, or 1:1 by volume. Any medically suitable form of collagen may be included in the composition, such as type I collagen, II collagen, or III collagen. The collagen may be derived from a mammalian source, such as a bovine or human source. The collagen fibrillar structure can be native, atelocollagen, hydrolyzed, or a combination of several types. Typically, no more than 10% of the collagen in the collagen scaffold demonstrates faster than alpha characteristics using gel electrophoresis.

A pharmaceutical composition containing PEP may be formulated in a variety of forms adapted to a preferred route of administration. Thus, a pharmaceutical composition can be administered via known routes including, for example, oral, parenteral (e.g., intradermal, transcutaneous, subcutaneous, intramuscular, intravenous, intraperitoneal, submucosal etc.), or topical (e.g., application to nervous tissue exposed during surgery, intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A pharmaceutical composition also can be administered via a sustained or delayed release. A pharmaceutical composition can be administered in the form of, for example, one or more injections, a suppository, a topical gel, a patch, or a non-surgical implant, such as a ring. A pharmaceutical composition may be administered embedded in a physical scaffold, such as a bandage, a suture, a staple, a splint, or surgical mesh. In one or more embodiments, multiple methods of administration may be utilized during a single treatment.

Thus, a pharmaceutical composition may be provided in any suitable form including but not limited to a solution, a suspension, an emulsion, a spray, an aerosol, or any form of mixture. The pharmaceutical composition may be delivered in formulation with any pharmaceutically acceptable excipient, carrier, or vehicle. For example, the formulation may be delivered in a conventional topical dosage form such as, for example, a cream, an ointment, an aerosol formulation, a suppository, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like. A formulation may be conveniently presented in unit dosage form and may be prepared by methods well known in the art of pharmacy. Methods of preparing a composition with a pharmaceutically acceptable carrier include the step of bringing the PEP into association with a carrier that constitutes one or more accessory ingredients. In general, a formulation may be prepared by uniformly and/or intimately bringing the PEP into association with a liquid carrier, a finely divided solid carrier, or both, and then, if necessary, shaping the product into the desired formulations.

The amount of PEP administered can vary depending on various factors including, but not limited to, the content and/or source of the PEP being administered, the weight, physical condition, and/or age of the subject, and/or the route of administration. Thus, the absolute weight of PEP included in a given unit dosage form can vary widely, and depends upon factors such as the species, age, weight, and physical condition of the subject, and/or the method of administration. Accordingly, it is not practical to set forth generally the amount that constitutes an amount of PEP effective for all possible applications. Those of ordinary skill in the art, however, can readily determine the appropriate amount with due consideration of such factors.

In one or more embodiments, a dose of PEP can be measured in terms of the PEP exosomes delivered in a dose. Thus, in one or more embodiments, the method can include administering sufficient PEP to provide a dose of, for example, from about 1×10⁶ PEP exosomes to about 1×10¹⁵ PEP exosomes to the subject, although in one or more embodiments the methods may be performed by administering PEP in a dose outside this range.

In one or more embodiments, therefore, the method can include administering sufficient PEP to provide a minimum dose of at least 1×10⁶ PEP exosomes, at least 1×10⁷ PEP exosomes, at least 1×10⁸ PEP exosomes, at least 1×10⁹ PEP exosomes, at least 1×10¹⁰ PEP exosomes, at least 1×10¹¹ PEP exosomes, at least 2×10¹¹ PEP exosomes, at least 3×10¹¹ PEP exosomes, at least 4×10¹¹ PEP exosomes, at least 5×10¹¹ PEP exosomes, at least 6×10¹¹ PEP exosomes, at least 7×10¹¹ PEP exosomes, at least 8×10¹¹ PEP exosomes, at least 9×10¹¹ PEP exosomes, at least 1×10¹² PEP exosomes, 2×10¹² PEP exosomes, at least 3×10¹² PEP exosomes, at least 4×10¹² PEP exosomes, or at least 5×10¹² PEP exosomes, at least 1×10¹³ PEP exosomes, or at least 1×10¹⁴ PEP exosomes.

In one or more embodiments, the method can include administering sufficient PEP to provide a maximum dose of no more than 1×10¹⁵ PEP exosomes, no more than 1×10¹⁴ PEP exosomes, no more than 1×10¹³ PEP exosomes, no more than 1×10¹² PEP exosomes, no more than 1×10¹¹ PEP exosomes, or no more than 1×10¹⁰ PEP exosomes.

In one or more embodiments, the method can include administering sufficient PEP to provide a dose characterized by a range having endpoints defined by any a minimum dose identified above and any maximum dose that is greater than the minimum dose. For example, in one or more embodiments, the method can include administering sufficient PEP to provide a dose of from 1×10¹¹ to 1×10¹³ PEP exosomes such as, for example, a dose of from 1×10¹¹ to 5×10¹² PEP exosomes, a dose of from 1×10¹² to 1×10¹³ PEP exosomes, or a dose of from 5×10¹² to 1×10¹³ PEP exosomes. In one or more certain embodiments, the method can include administering sufficient PEP to provide a dose that is equal to any minimum dose or any maximum dose listed above. Thus, for example, the method can involve administering a dose of 1×10¹⁰ PEP exosomes, 1×10¹¹ PEP exosomes, 5×10¹¹ PEP exosomes, 1×10¹² PEP exosomes, 5×10¹² PEP exosomes, 1×10¹³ PEP exosomes, or 1×10¹⁴ PEP exosomes.

Alternatively, a dose of PEP can be measured in terms of the concentration of PEP upon reconstitution from a lyophilized state. Thus, in one or more embodiments, the methods can include administering PEP to a subject at a dose of, for example, from about a 0.01% solution to a 100% solution to the subject, although in one or more embodiments the methods may be performed by administering PEP in a dose outside this range. As used herein, a 100% solution of PEP refers to one vial of PEP (approximately 2×10¹¹ exosomes or 75 mg) solubilized in 1 ml of a liquid or gel carrier (e.g., water, phosphate buffered saline, serum free culture media, surgical glue, tissue adhesive, etc.). For comparison, a dose of 0.01% PEP is roughly equivalent to a standard dose of exosomes prepared using conventional methods of obtaining exosomes such as exosome isolation from cells in vitro using standard cell conditioned media.

In one or more embodiments, therefore, the method can include administering sufficient PEP to provide a minimum dose of at least 0.01%, at least 0.05%, at least 0.1%, at least 0.25%, at least 0.5%, at least 1.0%, at least 2.0%, at least 3.0%, at least 4.0%, at least 5.0%, at least 6.0%, at least 7.0%, at least 8.0%, at least 9.0%, at least 10%, at least 15%, at least 20%, at least 25%, at least 30%, at least 35%, at least 40%, at least 45%, at least 50%, at least 60%, or at least 70%.

In one or more embodiments, the method can include administering sufficient PEP to provide a maximum dose of no more than 100%, no more than 90%, no more than 80%, no more than 70%, no more than 60%, no more than 50%, no more than 40%, no more than 30%, no more than 20%, no more than 10%, no more than 9.0%, no more than 8.0%, no more than 7.0%, no more than 6.0%, no more than 5.0%, no more than 4.0%, no more than 3.0%, no more than 2.0%, no more than 1.0%, no more than 0.9%, no more than 0.8%, no more than 0.7%, no more than 0.6%, no more than 0.5%, no more than 0.4%, no more than 0.3%, no more than 0.2%, or no more than 0.1%.

In one or more embodiments, the method can include administering sufficient PEP to provide a dose characterized by a range having endpoints defined by any a minimum dose identified above and any maximum dose that is greater than the minimum dose. For example, in one or more embodiments, the method can include administering sufficient PEP to provide a dose of from 1% to 50% such as, for example, a dose of from 5% to 20%. In one or more certain embodiments, the method can include administering sufficient PEP to provide a dose that is equal to any minimum dose or any maximum dose listed above. Thus, for example, the method can involve administering a dose of 0.05%, 0.25%, 1.0%, 2.0%, 5.0%, 20%, 25%, 50%, 80%, or 100%.

A single dose may be administered all at once, continuously for a prescribed period of time, or in multiple discrete administrations. When multiple administrations are used, the amount of each administration may be the same or different. For example, a prescribed daily dose of may be administered as a single dose, continuously over 24 hours, as two administrations, which may be equal or unequal. When multiple administrations are used to deliver a single dose, the interval between administrations may be the same or different. In certain embodiments, PEP may be administered as a once-off administration, for example, during a surgical procedure.

In certain embodiments in which multiple administrations of the PEP composition are administered to the subject, the PEP composition may be administered as needed to treat damaged vaginal tissue to the desired degree. Alternatively, the PEP composition may be administered twice, three times, four times, five times, six times, seven times, eight times, nine times, or at least ten times. The interval between administrations can be a minimum of at least one day such as, for example, at least three days, at least five days, at least seven days, at least ten days, at least 14 days, or at least 21 days. The interval between administrations can be a maximum of no more than six months such as, for example, no more than three months, no more than two months, no more than one month, no more than 21 days, or no more than 14 days.

In one or more embodiments, the method can include multiple administrations of PEP to a subject at an interval (for two administrations) or intervals (for more than two administrations) characterized by a range having endpoints defined by any minimum interval identified above and any maximum interval that is greater than the minimum interval. For example, in one or more embodiments, the method can include multiple administrations of PEP at an interval or intervals of from one day to six months such as, for example, from three days to ten days. In certain embodiments, the method can include multiple administrations of PEP at an interval of that is equal to any minimum interval or any maximum interval listed above. Thus, for example, the method can involve multiple administrations of PEP at an interval of three days, five days, seven days, ten days, 14 days, 21 days, one month, two months, three months, or six months.

In one or more embodiments, the methods can include administering a cocktail of PEP that is prepared from a variety of cell types, each cell type having a unique epithelial growth profile—e.g., protein composition and/or gene expression. In this way, the PEP composition can provide a broader spectrum of activity promoting growth of epithelial tissues than if the PEP composition is prepared from a single cell type.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

Reference throughout this specification to “one embodiment,” “an embodiment,” “certain embodiments,” “some embodiments,” or “one or more embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, particular embodiments may be described in isolation for clarity. Thus, unless otherwise expressly specified that the features of a particular embodiment are incompatible with the features of another embodiment, the particular features, configurations, compositions, or characteristics may be combined in any suitable manner in one or more embodiments. Thus, features described in the context of one embodiment may be combined with features described in the context of a different embodiment except where the features are necessarily mutually exclusive.

The words “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

For any method disclosed herein that includes discrete steps, the steps may be conducted in any feasible order. And, as appropriate, any combination of two or more steps may be conducted simultaneously.

### EXEMPLARY EMBODIMENTS

Embodiment 1 is a method for treating exposure of surgical mesh in a subject, the method comprising:
   administering PEP to tissue adjacent to an area of exposed surgical mesh in an amount effective to treat the area of exposed surgical mesh.
Embodiment 2 is a method for treating exposure of surgical mesh in a subject, the method comprising:
   surgically closing an area of exposed surgical mesh; and
   administering PEP to epithelium adjacent to the surgical closure in an amount effective to treat the area of exposed surgical mesh.
Embodiment 3 is the method of Embodiment 1 or Embodiment 2, wherein the amount effective to treat the area of exposed surgical mesh comprises an amount effective to increase proliferation of epithelium to decrease the area of exposed surgical mesh, increase thickness of epithelium covering the surgical mesh, or increase vascularization of epithelium covering the surgical mesh.
Embodiments 4 is the method of any preceding Embodiment, wherein the PEP is administered once.
Embodiment 5 is the method of any one of Embodiments 1-3, wherein the PEP is administered more than once.
Embodiment 6 is the method of Embodiment 5, wherein the PEP is provided in from 2 to 4 administrations.
Embodiment 7 is the method of Embodiment 5 or Embodiment 6, wherein the PEP is administered at an interval of about seven days between administrations.
Embodiment 8 is the method of any preceding Embodiment, wherein PEP is administered in an amount effective to deliver at least 10¹² PEP exosomes.
Embodiment 9 is the method of any preceding Embodiment, wherein the surgical mesh is at least 1 cm by 1 cm in size.
Embodiment 10 is the method of any one of Embodiments 2-9, wherein the method results in mesh exposure resolution as measured by increased cellular proliferation, increased epithelial thickness, increased vascularization, or lack of dehiscence compared to an untreated mesh exposure.
Embodiment 11 is the method of any preceding Embodiment, wherein the method results in reduced scar formation as measured by tissue microscopy compared to an untreated mesh exposure.
Embodiment 12 is the method of any preceding Embodiment, wherein administering PEP comprises injecting PEP at one or more sites within the treated area.
Embodiment 13 is the method of any preceding Embodiment, wherein the PEP is administered in a composition that further comprises an extracellular matrix component.
Embodiment 14 is the method of Embodiment 13, wherein the extracellular matrix component comprises collagen.

### EXAMPLES

The present invention is illustrated by the following examples. It is to be understood that the particular examples, materials, amounts, and procedures are to be interpreted broadly in accordance with the scope and spirit of the invention as set forth herein.

### Example 1

A porcine model was used due to the similarity between porcine and human anatomy and because the vaginal caliber and length allow for multiple clinically meaningfully-sized meshes to be implanted per animal.

### Mesh Exposure Model

Sixteen macroporous monofilament polypropylene meshes (RESTORELLE M Flat Mesh; Coloplast Corp., Minneapolis, MN) were surgically implanted in the vaginas of seven domestic Yorkshire-Crossed pigs, weighing 70-80 kg. Animals were divided into two groups: (Group 1) vaginal epithelial re-closure with (N=2) or without (N=2) PEP injection, and (Group 2) injection of PEP only (N=3).

The mesh exposure model was created by denuding the vaginal epithelium and underlying fibromuscular tissue with electrocautery then affixing mesh squares to this area with interrupted 2-0 polypropylene suture. For Group 1, a 3 cm by 3 cm mesh was implanted on the ventral and rostral aspect of the vagina. For Group 2, different sized meshes (1 cm by 1 cm, 2 cm by 2 cm, 3 cm by 3 cm) were implanted circumferentially (FIG. 1). After 7-10 days, the animal returned to the operating room for the intervention procedure.

### Preparation of Purified Exosome Product (PEP)

PEP was prepared as previously described (International Patent Application NO. PCT/US2018/065627, which published as International Publication No. WO 2019/118817).

The PEP was stored at room temperature until reconstituted for use.

Intervention Procedure with or without Purified Exosome Product

Groups underwent surgical, exosome, or combined treatment for their mesh exposures. For Groups 1A and 1B, the epithelium and underlying fibromuscular tissue adjacent to the mesh were mobilized and reapproximated, tension-free, with 2-0 polyglactin 910 suture in an interrupted fashion. Group 1B had a concomitant injection of 2.5 mL of reconstituted PEP, deep to the mesh and closure, evenly across the area encompassed by the mesh. Group 2 underwent PEP injection only; PEP (2.5 mL) was injected below the mesh, into the vaginal tissues as described above.

A 20% PEP gel was created by reconstituting 5×10¹² lyophilized PEP exosomes (Rion LLC, Rochester, MN) in 1 mL of sterile water and 4 mL of clinical grade type I bovine collagen (5 mg/mL; Collagen Solutions, Glasgow, UK) and injected using a 21-gauge needle.

2′-Deoxy-5-ethynyluridine (EdU) is incorporated into DNA, as a thymidine analog, during DNA replication. It can be used to track tissue regeneration. It was given as an oral supplement (5 mg/kg) twice weekly to one animal in the PEP-only group.

### Wildtype

Un-instrumented female pigs of equivalent size and age were used for vaginal biopsy and comparison as the “wildtype.” Post-sacrifice processing was carried out in a similar fashion to the experimental groups.

### Sacrifice and Tissue Harvest

Four weeks after the intervention, animals were euthanized by pentobarbital. The genitourinary organs were removed en-bloc and photographed. The center-most portion of regenerated tissue from each mesh complex was sampled using a scalpel. If no regeneration was noted, a peripheral biopsy was taken adjacent to the mesh edge. Tissue specimens were then placed in cassettes and bathed in formalin or Trump's fixative solution.

### Histologic Processing and Quantification

Excised samples were processed for transmission electron microscopy (TEM), Hematoxylin and Eosin (H&E), Masson's Trichrome staining, and immunohistochemistry.

Formalin fixed samples were embedded in paraffin and cut on a microtome into 10 μm sections. Sections were subjected to standard histopathological analysis including Hematoxylin and Eosin (H&E) and Masson's Trichrome staining (FIG. 3A). H&E-stained sections were imaged using a slide scanner (AXIO SCAN.Z1, Carl Zeiss Microscopy GmbH, Jena, Germany). For immunohistochemical analysis, sections were deparaffinized by serial washes in xylene and re-hydrated in decreasing amounts of ethanol. Antigen retrieval was performed by immersing sections in sodium citrate buffer (10 mM sodium citrate, 0.05% Tween 20, pH 6.0) and boiling for 30 minutes. Sections were outlined with a hydrophobic pen, blocked with blocking buffer (PBS with 5% normal donkey serum, 5% bovine serum albumin, 0.2% Triton-X) for one hour at room temperature, and incubated with primary antibodies (Table 1) diluted in blocking buffer overnight at 4° C. ALEXA FLUOR (Molecular Probes, Inc., Eugene, OR) secondary antibodies (donkey) were diluted 1:500 in blocking buffer and incubated for one hour at room temperature. EdU was labeled using an imaging kit (CLICK-IT PLUS EdU AF647, Thermo Fisher Scientific, Inc., Waltham, MA) according to the manufacturer's instructions. Following extensive washes, ProLong Gold Antifade Mountant with DAPI was added onto each section, covered with a cover glass, and imaged on an inverted fluorescent microscope with a 10× objective (AXIO OBSERVER, Carl Zeiss Microscopy GmbH, Jena, Germany).

TABLE 1
Primary Antibodies

| 1° Antibody | Company | Catalog # | Species | Dilution |
|---|---|---|---|---|
| SMA (smooth | Abcam | ab7817 | Mouse | 1:500 |
| muscle actin) | | | | |
| CD31 | Abcam | ab28364 | Rabbit | 1:50 |
| Collagen III | SouthernBiotech | 1330-01 | Goat | 1:10 |
| Pan-cytokeratin | Abcam | Ab86734 | Mouse | 1:200 |
| E-Cadherin | Proteintech | 60335-1-lg | Mouse | 1:500 |
| Ki67 | Abcam | Ab15580 | Rabbit | 1:500 |
| CD3 | Abcam | Ab11089 | Rat | 1:200 |

Average epithelial thickness was measured from H&E-stained sections. Area of epithelium was calculated by manual outlining in FIJI (v.2.0) then divided by the length of epithelium. Immunofluorescence was quantified using FIJI equipped with GDSC plug-in and custom macros using thresholding, mask creation, and particle analysis. Degree of proliferation was assessed by counting the number of Ki67⁺ cells in a given 10× field of view per length of epithelium. A mask was created using an epithelial marker (Pan-cytokeratin) to quantify the number of total and epithelial Ki67⁺ cells per length of epithelium. Non-epithelial proliferation was calculated as the difference between total and epithelial Ki67⁺ cells. Vascularization was assessed by quantifying the number of CD31⁺ blood vessel lumens per mm² of sub-epithelial area. Degree of co-distribution of CD31⁺ and SMA⁺ regions was used as a surrogate for smooth muscle deposition outside of blood vessels, and was reported as an intensity correlation quotient (ICQ). Chronic inflammation was assessed by measuring the percent area covered by CD3⁺ cells (T lymphocytes) per high-power field. Histologic processing and quantification were performed by a blinded investigator.

### Ultrasound Vibroelastography

To evaluate tissue biomechanics in vivo, elastography was performed prior to sacrifice. A 0.1-s harmonic vibration (100 Hertz) was generated on the perineum using a handheld vibrator (Model FG-142, Labworks Inc., Costa Mesa, CA) as previously described (Zhou et al., 2019. Radiology 291 (2): 479-484). The excitation signal was amplified by an audio amplifier (Model D150A, Crown Audio Inc., Elkhart, IN) as previously described (Zhou et al., 2017. Ultrasonics 81 (Supplement C): 86-92). Wave propagation in the vagina was measured using a linear array ultrasound probe (L11-5v, Verasonics, Inc., Kirkland, WA), with a central frequency of 6.4 MHz, on an ultrasound system (Verasonics, Inc., Kirkland, WA).

Speed maps were obtained and a representative shear wave speed was reported for each mesh. Tissue viscosity was obtained using post-collection computation. The radiofrequency data of the ultrasound echo were obtained and demodulated to get IQ data of the ultrasound signals. The tissue motion was obtained by using auto-correlation analysis of the ultrasound IQ data as previously described (Zhang et al., 2017. IEEE Trans Ultrason Ferroelectr Frequency Control 64 (9): 1298-1304). With cross-correlation technique, the wave speed in both the horizontal and vertical directions was measured and the speed map of the region of interest obtained, as previously described (Zhang et al., 2019. IEEE Trans Ultrason Ferroelectr Frequency Control 66 (5): 1346-1352).

The measurement of wave speeds at multiple frequencies can be used to calculate the shear elasticity and shear viscosity with the Voigt's model,

$${c}_{s} = \sqrt{\frac{2 ( {μ}_{1}^{2} + {ω}^{2} {μ}_{2}^{2} )}{ρ ( {μ}_{1} + \sqrt{{μ}_{1}^{2} + {ω}^{2} {μ}_{2}^{2}} )}}$$

where ω is the angular frequency, μ₁ is the shear elasticity, and μ₂ is the shear viscosity.

Shear elasticity and viscosity were identified via Levenberg-Marquardt nonlinear, least-square algorithm by minimizing the difference between measured and predicted shear wave speeds at different vibration frequencies, as previously described (Prim et al., 2016. J Mec Behav Biomed Mater 54:93-105). Residual error of the objective function was calculated as:

$$Ω = {\sum }_{n = 1}^{N} {( {c}_{n}^{T} - {c}_{n}^{E} )}^{2}$$

where the superscripts E and T refer to the experimentally measured and theoretically predicted values of shear wave speed, and subscript n indicates a particular experimental state.

Average wave speeds and viscosity were similar amongst the groups. The findings suggest that PEP does not cause fibrosis predominant wound healing which would lead to unfavorable viscoelastic changes.

### Statistical Analysis

Given the pilot nature of this study, limited statistical analysis was performed. Quantifiable data were reported as mean±standard deviation. Where appropriate, data were analyzed with one-way ANOVA and post-hoc Tukey's test, with a significance level set to a of 0.05.

### Example 2

### Experimental Design

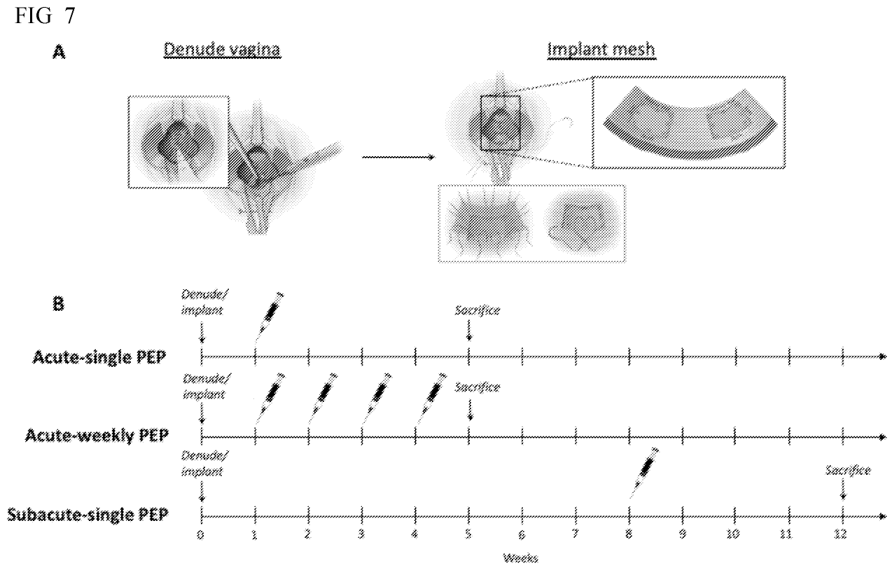

Animals were divided into three groups based on timing and frequency of PEP injection. Group 1: single PEP injection seven days after mesh implantation (acute-single, N=1, 2 meshes). Group 2: weekly PEP injection, beginning seven days after mesh implantation and continuing weekly for a total of four weekly injections (acute-weekly, 4 injections, N=2, 4 meshes). Group 3: delayed single PEP injection eight weeks after mesh implantation (subacute-single, N=3, 6 meshes). More animals were devoted to the subacute group to account for possible animal and tissue losses over a longer duration. Fewer animals were placed in the control group (acute-single) due to homogeneous data from Example 1. All groups were sacrificed four weeks after initial PEP injection (FIG. 7). None of the groups received concomitant surgical closure given prior data suggesting PEP's efficacy as a non-surgical therapy for mesh exposure (Example 1, FIGS. 1-6). All animals were dosed with 2′-deoxy-5-ethynyluridine (EdU, Carbosynth LLC, San Diego, CA), a thymidine analog that gets incorporated into replicating DNA, at a dose of 5 mg/kg (10-20 mL orally, twice weekly beginning on the day of PEP injection) to track cellular proliferation.

### Mesh Exposure Model

The mesh exposure model was created using previously described methods (Example 1). Briefly, six domestic Yorkshire-Crossed pigs (70-80 kg) were anesthetized and prepped with sterile technique. Betadine was applied intravaginally and across the perineum and hind legs. Vaginal epithelium and fibromuscular tissue were mobilized and excised in a 3 cm by 3 cm area, per usual surgical technique with electrocautery. Macroporous monofilament polypropylene mesh (RESTORELLE M Flat Mesh; Coloplast Corp., Minneapolis, MN), was cut to 3 cm by 3 cm sections and affixed to the denuded regions in the mid-ventral and mid-rostral vagina (2 meshes implanted per pig) using interrupted 2-0 polyglactin 910 sutures along the lateral edges.

### Injection of Purified Exosome Product (PEP)

A working solution of 20% PEP was created by reconstituting 5×10¹² lyophilized PEP exosomes (Rion LLC, Rochester, MN) in 1 mL of sterile water and 4 mL of clinical grade type I bovine collagen (5 mg/mL; Collagen Solutions, Glasgow, UK). After reconstitution, the PEP solution formed a gel-like consistency. Following a sterile betadine preparation, a total of 2.5 mL of PEP gel was injected into the fibromuscular tissue under each mesh exposure, evenly across nine sites (in a 3×3 grid pattern) using a 21-gauge needle.

### Tissue Harvest and Histologic Processing

Four weeks following the initial PEP injection, all animals were euthanized using pentobarbital. Genitourinary organs were excised en-bloc and photographed. Subsequently, the center-most portion of regenerated vagina-mesh complexes was sampled using a scalpel, placed into cassettes and immersed in formalin. Formalin-fixed samples were embedded in paraffin and cut on a microtome into 10-μm sections. Sections were processed for hematoxylin and eosin (H&E) staining and immunohistochemistry according to standard lab protocols. H&E-stained sections were imaged using a 10× objective on a MOTIC EASYSCAN Pro digital slide scanner (Motic Ltd., Hong Kong).

For immunohistochemical analysis, sections were deparaffinized with serial washes in xylene and re-hydrated in decreasing amounts of ethanol. Antigen retrieval was performed by immersing sections in sodium citrate buffer (10 mM sodium citrate, 0.05% Tween 20, pH 6.0) and boiling for 30 minutes. Sections were outlined with a hydrophobic pen, blocked with blocking buffer (PBS with 5% normal donkey serum, 5% bovine serum albumin, 0.2% Triton-X) for one hour at room temperature, and incubated with primary antibodies (Table 1) diluted in blocking buffer overnight at 4° C. ALEXA FLUOR (Molecular Probes, Inc., Eugene, OR) secondary antibodies (raised in donkey) were diluted 1:500 in blocking buffer and incubated for one hour at room temperature. EdU was labeled using the CLICK-IT PLUS EdU AF647 imaging kit (Thermo Fisher Scientific, Inc., Waltham, MA) according to the manufacturer's instructions after completing secondary antibody labeling. Following extensive washes, PROLONG Gold (Thermo Fisher Scientific, Inc., Waltham, MA) antifade mountant with DAPI (4′,6-diamidino-2-phenylindole) was added onto each section, covered with a cover glass, and imaged on an inverted fluorescent microscope with a 10× objective (AXIO OBSERVER, Carl Zeiss Microscopy GmbH, Jena, Germany).

TABLE 1
Primary antibodies for immunofluorescence

| 1° Antibody | Company | Catalog # | Species | Dilution |
|---|---|---|---|---|
| CD31 | Abcam | ab28364 | Rabbit | 1:50 |
| Pan-cytokeratin | Abcam | ab86734 | Mouse | 1:200 |

### Quantification and Statistical Analysis

Epithelial thickness was calculated from entire sections that were automatically stitched from individual images obtained by the slide scanner. Using FIJI (v. 3.0; Schindelin et al., 2012, Nature Methods 9 (7): 676-682), both the area and length of the epithelium in the whole section were manually traced, and average thickness was reported as the ratio of area to length of epithelium. Cellular proliferation was measured using a semi-automated custom macro written in FIJI. Briefly, a mask outlining the epithelial area (positive for Cytokeratin) was applied to immunofluorescence images co-staining cytokeratin, EdU, and DAPI. The number of EdU⁺ nuclei was counted within the mask (epithelial proliferation), outside of the mask (non-epithelial), and across the entire image (total proliferation). Since EdU⁺ cells are present throughout the entire thickness of the epithelium, proliferation was normalized to the area of the analyzed epithelium. Finally, a manual dual-counter was applied to immunofluorescence images co-staining cytokeratin, CD31, EdU, and DAPI. Capillary density was reported as the number of CD31⁺ vessel lumens per area of sub-epithelium (cytokeratin negative area), and proliferative capillaries were reported as the number of CD31⁺ vessels that exhibited at least one EdU⁺ nucleus. Data were reported as mean ⁺/−standard error. Where appropriate, data were analyzed with ANOVA and post-hoc Tukey's test. A significance level was set to a of 0.05.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method for treating exposure of surgical mesh in a subject, the method comprising:
administering PEP to tissue adjacent to an area of exposed surgical mesh in an amount effective to treat the area of exposed surgical mesh.

2. A method for treating exposure of surgical mesh in a subject, the method comprising:
surgically closing an area of exposed surgical mesh; and
administering PEP to epithelium adjacent to the surgical closure in an amount effective to treat the area of exposed surgical mesh.

3. The method of claim 1, wherein the amount effective to treat the area of exposed surgical mesh comprises an amount effective to increase proliferation of epithelium to decrease the area of exposed surgical mesh, increase thickness of epithelium covering the surgical mesh, or increase vascularization of epithelium covering the surgical mesh.

4. The method of claim 1, wherein the PEP is administered once.

5. The method of claim 1, wherein the PEP is administered more than once.

6. The method of claim 5, wherein the PEP is provided in from 2 to 4 administrations.

7. The method of claim 5, wherein the PEP is administered at an interval of about seven days between administrations.

8. The method of claim 1, wherein PEP is administered in an amount effective to deliver at least 10¹² PEP exosomes.

9. The method of claim 1, wherein the surgical mesh is at least 1 cm by 1 cm in size.

10. The method of claim 2, wherein the method results in mesh exposure resolution as measured by increased cellular proliferation, increased epithelial thickness, increased vascularization, or lack of dehiscence compared to an untreated mesh exposure.

11. The method of claim 1, wherein the method results in reduced scar formation as measured by tissue microscopy compared to an untreated mesh exposure.

12. The method of claim 1, wherein administering PEP comprises injecting PEP at one or more sites within the treated area.

13. The method of claim 1, wherein the PEP is administered in a composition that further comprises an extracellular matrix component.

14. The method of claim 13, wherein the extracellular matrix component comprises collagen.

15. The method of claim 2, wherein the amount effective to treat the area of exposed surgical mesh comprises an amount effective to increase proliferation of epithelium to decrease the area of exposed surgical mesh, increase thickness of epithelium covering the surgical mesh, or increase vascularization of epithelium covering the surgical mesh.

16. The method of claim 2, wherein the PEP is administered once.

17. The method of claim 2, wherein the PEP is administered more than once.

18. The method of claim 17, wherein the PEP is provided in from 2 to 4 administrations.

19. The method of claim 17, wherein the PEP is administered at an interval of about seven days between administrations.

20. The method of claim 2, wherein PEP is administered in an amount effective to deliver at least 10¹² PEP exosomes.

21. The method of claim 2, wherein the surgical mesh is at least 1 cm by 1 cm in size.

22. The method of claim 2, wherein the method results in reduced scar formation as measured by tissue microscopy compared to an untreated mesh exposure.

23. The method of claim 2, wherein administering PEP comprises injecting PEP at one or more sites within the treated area.

24. The method of claim 2, wherein the PEP is administered in a composition that further comprises an extracellular matrix component.

25. The method of claim 24, wherein the extracellular matrix component comprises collagen.
